(19) United States
(12) Patent Application Publication
(10) Pub. No.: US 2026/0071937 A1
(43) Pub. Date: Mar. 12, 2026

(54) Autonomous Sediment Core Sampler System and Method

(71) Applicants: Woods Hole Oceanographic Institution, Woods Hole, MA (US); California Institute of Technology on behalf of Jet Propulsion Laboratory, Pasadena, CA (US)

(72) Inventors: Casey Machado, Woods Hole, MA (US); Timothy Shank, Woods Hole, MA (US); Francesca Daszak, Woods Hole, MA (US); Victor Naklicki, Woods Hole, MA (US); Andrew Klesh, Pasadena, CA (US); Russell Smith, Pasadena, CA (US); Joel Steinkraus, Pasadena, CA (US)

(21) Appl. No.: 19/322,035

(22) Filed: Sep. 8, 2025

Related U.S. Application Data
(60) Provisional application No. 63/692,407, filed on Sep. 9, 2024.

(51) Int. Cl. G01N 1/08; G01N 33/24

(52) U.S. Cl. CPC G01N 1/08; G01N 33/24

(57) ABSTRACT

A system and method for obtaining a sediment core sample utilizing a stationary frame assembly including a first drive mechanism, and at least one traveling frame assembly movable in a first direction relative to the stationary frame and carrying at least one core tube and carrying a second drive mechanism for each core tube. The first drive mechanism, activated by a controller, is configured to advance the traveling frame in the first direction until a selected parameter is achieved. The second drive mechanism is configured to drive the core tube to a fully extended position in the first direction, when the selected parameter is achieved, by applying a constant force while driving the core tube to the fully extended position. An occlusion mechanism blocks the opening of each core tube after the core tube is removed from the sediment.

### CROSS-REFERENCE TO RELATED APPLICATION

This application claims priority to U.S. Provisional Application No. 63/692,407 filed 9 Sep. 2024. The entire contents of the above-mentioned application are incorporated herein by reference.

### STATEMENT REGARDING GOVERNMENT LICENSE RIGHTS

The invention described herein was made with U.S. government support under Grant No. 80NMO0018D0004 awarded by NASA (JPL). The U.S. Government has certain rights in the invention.

### FIELD OF THE INVENTION

This invention relates to systems having at least two types of core tube advancement mechanism for obtaining sediment core samples.

### BACKGROUND OF THE INVENTION

Collecting sediment samples from the bottom of a liquid environment such as found in oceans, lakes, and rivers and other bodies of water, is a necessary step to understanding the composition, structure, and history of sediment deposits. In environmental studies, sediment collections are analyzed for pollutants, contaminants, and chemical compositions to assess environmental health. Geological research uses sediment samples to understand the various deposit layers and their history in the context of geological processes, climate change, and natural hazards. Biologists have long used sediment samples to examine the composition and distribution of size classes and properties of fauna, microorganisms and their micro-environment to study the ecology and evolution of life.

Currently, sediment sampling methods in aquatic environments include grab samplers, tethered multi-core samplers, suction samplers, and dredges that often include the use of tethered or human occupied submergence vehicles. Grab samplers, such as Van Veen grab samplers and Ekman grab samplers, scoop or “grab” a sample near the water-sediment interface. Dredging requires tethered equipment to drag across the surface and focuses on gathering a large quantity of sediment over a broad area.

Core samplers penetrate the surface of the sediment to collect a vertical section of sediment or “core”; existing systems include gravity corers, piston corers, and box corers. Existing sediment core sampling designs require the ability to generate large reaction forces either through weight, velocity, or thrust, to oppose the force of core tube insertion. Additionally, all existing solutions require the collection vehicle to apply a large magnitude reaction force therefore making this type of sampling difficult or impossible to do from smaller, lower mass, vehicles not capable of producing these forces efficiently, despite these smaller vehicles being much greater economically efficient platforms for surveying and sampling, especially for problems that require scalable solutions.

Lastly, remotely operated vehicles (ROVs) and human-occupied vehicles (HOVs) have been coupled to sampling systems for more precise sample collection at depth. For example, the ability to take sediment core samples with this precision has previously required use of a large vehicles such as ROV Jason or HOV Alvin, equipped with a hydraulic manipulator arm and operated by a skilled pilot. These platforms represent significantly higher costs and substantial infrastructure to support.

The expansive use of sediment samples across multiple industries, such as oceanographic research, oil and gas, mining, fisheries, among others, has prompted discussion of increased regulations. Many emerging markets such as exclusive economic zone (EEZ) exploration, seafloor mineral prospecting regulation, genetic information sharing regulations and guidelines, bioprospecting regulation, and wind farm operation regulation all will likely mandate sampling such as sediment cores to enforce environmental regulatory information gathering for analysis of effects on biodiversity and beyond. The International Seabed Authority published “Draft guidelines for the establishment of baseline environmental data” with respect to seafloor mining operations, in which sediment cores are identified as a required type of sampling for several different scientific disciplines to measure a wide variety of variables. Additionally, the temporal and spatial requirements for sampling in this application lend themselves to solutions that employ multiple smaller autonomous underwater vehicles (AUVs) as an alternative to using a single ROV to attempt the same coverage.

Sediment cores have primarily been attained by use of ROVs or HOVs utilizing a remotely operated manipulator arm with high degrees of freedom. This requires large vehicles and operational support infrastructure. Additionally, cores are attained through drop core systems which are lowered to the seafloor from a ship via cable/winch, impact the bottom collect samples and are hauled back to the ship. These drop cores are only crudely targetable and have a reasonably high failure rate.

Among the research community, sediment core samples were identified as the greatest enabler to address and test leading edge scientific hypotheses, regardless of discipline (micro-and macro-biology, chemistry, geology). Whether it was for microbiology, pore-water geochemistry, carbon pathways, food-webs and metabolism, energetics, biodiversity, biogeography, genomics, productivity, sediment samples could be used by all disciplines to provide impactful samples. Therefore, a new sediment coring system that could operate from a modest AUV like Orpheus (550 lbs., 5′×6′ footprint) and other small vessels of opportunity would be transformative for many of the listed uses and industries.

### SUMMARY OF THE INVENTION

The present invention utilizes a novel sampler system attachable to a vehicle and configured to position a core tube and apply forces necessary to insert the core tube into sediment without compromising the integrity of the sample inside or relying on the vehicle carrying the sampler system to produce large reaction forces to hold the assembly in place. Existing designs for similar systems have failed to simultaneously address both these driving requirements, leading to significant limitations in their usage and sample quality.

This invention features a system and method for obtaining a sediment core sample utilizing a stationary frame assembly including a first drive mechanism, and at least one traveling frame assembly movable in a first direction relative to the stationary frame and carrying at least one core tube and carrying a second drive mechanism for each core tube. The first drive mechanism is configured to advance the traveling frame in the first direction until a selected parameter is achieved. The second drive mechanism is configured to drive the core tube to a fully extended position in the first direction when the selected parameter is achieved, and the second drive mechanism applies a constant force while driving the core tube to the fully extended position. An occlusion mechanism blocks the opening of each core tube after the core tube is removed from the sediment. A controller activates the first drive mechanism to initiate obtaining the core sample.

In some embodiments, the first drive mechanism includes a linear screw drive and the second drive mechanism includes at least one constant-force spring. In certain embodiments, the controller monitors the selected parameter as at least one of (i) a number of revolutions of the first drive mechanism and/or (ii) duration of operation of the first drive mechanism.

In some embodiments, the occlusion mechanism includes a core catcher assembly having a rotatable plate with a stopper for each core tube. In a number of embodiments, the controller detects when each core tube has been fully extended and commands the first drive mechanism to withdraw the core tube from the sediment in a second direction opposite to the first direction, and then commands the occlusion mechanism to block the opening of that core tube by moving the occlusion mechanism from a first position into a second position. That core tube is then driven in the first direction to block that opening with the respective stopper.

In certain embodiments when the system is attached to a vehicle, at least one of the sampler system and the vehicle include at least one tilt detection sensor and, when the tilt sensor detects that the vehicle is tilting beyond a selected amount, the controller triggers the second drive mechanism such as a pair of constant-force springs per core tube. In other embodiments, when the tilt sensor detects that the vehicle is tilting beyond a selected amount at a first sampling location, the controller stops sampling and requests repositioning to another sampling location.

This invention also features a method for obtaining a sediment core sample, including selecting a sampler system having a stationary frame assembly including a first linear drive mechanism and at least one traveling frame assembly movable in a first direction relative to the stationary frame and carrying at least one core tube and carrying a second drive mechanism for each core tube. The method further includes activating the first drive mechanism to advance the traveling frame in the first direction until a selected parameter is achieved and, after the selected parameter is achieved, utilizing the second drive mechanism to drive the core tube to a fully extended position in the first direction by applying a constant force while driving the core tube to the fully extended position. The opening of each core tube is then blocked after the core tube is removed from the sediment.

In some embodiments, the method further includes attaching the sampler system to a vehicle and deploying the vehicle with sampler system in a liquid environment, and the second drive mechanism includes at least one constant-force spring loaded firing mechanism to drive the core to its full insertion depth using the inertia of the vehicle to react against rather than the vehicle's ability to push back statically through weight or thrust.

In certain embodiments, the method includes blocking the opening of each core tube by rotating a push plate to align an elastomeric stopper directly below each filled core tube, and utilizing the linear drive mechanism to drive the bottom of the core tubes into the elastomeric stoppers to plug them closed and prevent sediment from escaping during the remainder of deployment and recovery.

### BRIEF DESCRIPTION OF THE DRAWINGS

To enable a better understanding of the present invention, and to show how the same may be carried into effect, certain embodiments of the invention are explained in more detail with reference to the drawings, by way of example only, in which:

FIG. 1 is a schematic front view of one embodiment of the present sediment sampler system wherein left and right core tubes have been triggered utilizing release burn wires and are in the fully “inserted”position;

FIG. 2 is a schematic perspective view of another embodiment of the overall sediment sampler system utilizing levers as trigger mechanisms;

FIGS. 3B-3D depict the full assembly of FIG. 2 from a front view (FIG. 3B), from a side view (FIG. 3C), and from a bottom view (FIG. 3D); and

FIGS. 4A-4C depict the first actuator assembly in FIG. 2 from a cross-section view (FIG. 4A), a side view (FIG. 4B), and a top-down view (FIG. 4C).

### DETAILED DESCRIPTION OF THE PRESENTLY PREFERRED EMBODIMENTS

The present invention was developed to enable autonomous collection of high-quality sediment core samples comparable to conventionally accepted methodologies already employed today. It has relevance to any application where sediment core samples are valuable (a fundamental interdisciplinary sample type for benthic oceanography and sediment structure analysis). Additionally, this design enables sediment coring samples to take place from small, agile autonomous vehicles which generally lack the ability to generate large reaction forces required to enable direct insertion due to their neutral buoyancy and relatively low power vertical thrust capacity. The quality of samples is comparable to ROV/HOV manually collected samples but is better than drop core systems while reducing the cost per core sample significantly when applied at scale on multiple vehicles.

The term “vehicle” is utilized herein in its broadest sense to include AUVs, ROVs, subsea landers (including free-falling dropped platforms with no propulsion), extraterrestrial landers, and exploratory vehicles of all kinds, with or without propulsion. The present invention is particularly suitable for environments in which gravity is lower than normal earth gravity or in liquid environments where buoyancy of the vehicle is near neutral buoyancy.

The present invention may be accomplished by a sediment core sampling assembly, also referred to herein as an Autonomous Sediment Core Sampler, that sequentially utilizes two insertion mechanisms to enable insertion without the need to generate large reaction forces. A first linear actuator such as a lead screw-driven initial insertion mechanism inserts the core tube as far as the softness of the sediment allows or to an initial insertion depth, depending on the parameter selected to transition from the first drive mechanism to the second drive mechanism. After a selected parameter is achieved, the system triggers a second drive mechanism such as a constant-force spring-loaded firing mechanism to drive the core to its full insertion depth using the inertia of the vehicle to react against rather than the vehicle's ability to push back statically through weight or thrust. An occlusion mechanism such as a push plate then passively rotates on its torsion spring to align elastomeric stoppers directly below filled core tubes, and the lead screw then drives the bottom of the core tubes into the stoppers to plug them closed and prevent sediment from escaping during the remainder of the deployment and recovery.

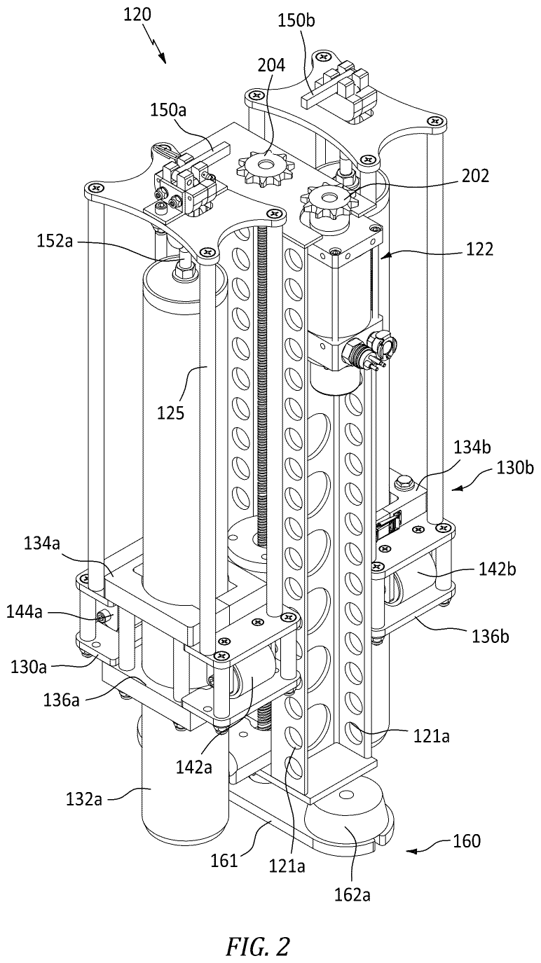

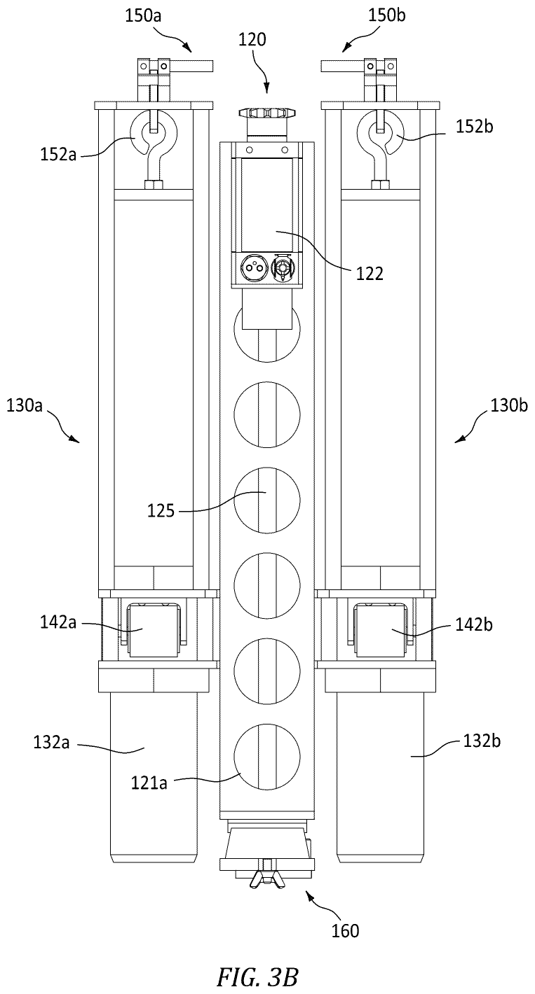

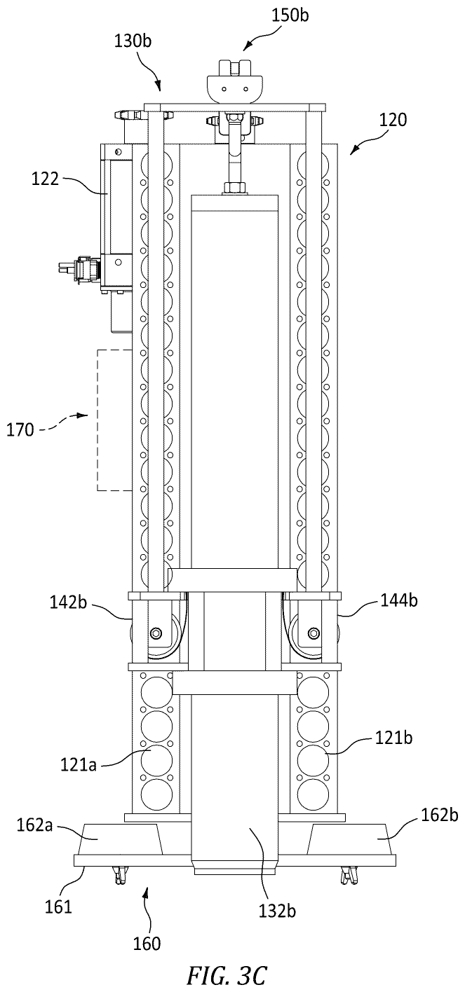

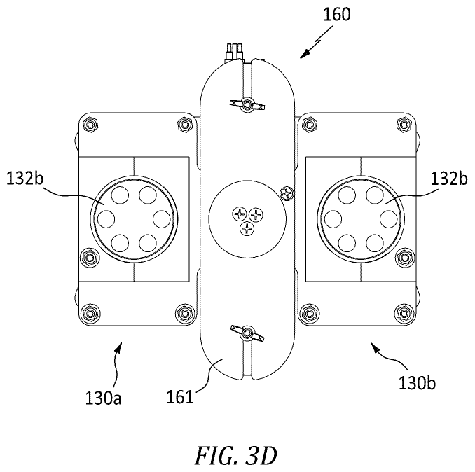

As described in more detail below, in a number of constructions, the parameter selected according to the present invention directly or indirectly measures initial travel of the core tubes, such as by counting the number of revolutions of the first drive motor, the number of revolutions of the lead screw, or counting duration of initial actuation utilizing a clock or other timing mechanism. A certain degree of tilt is utilized in other constructions. Alternatively, a mechanical trigger is activated such as described below in relation to FIGS. 2-3C.

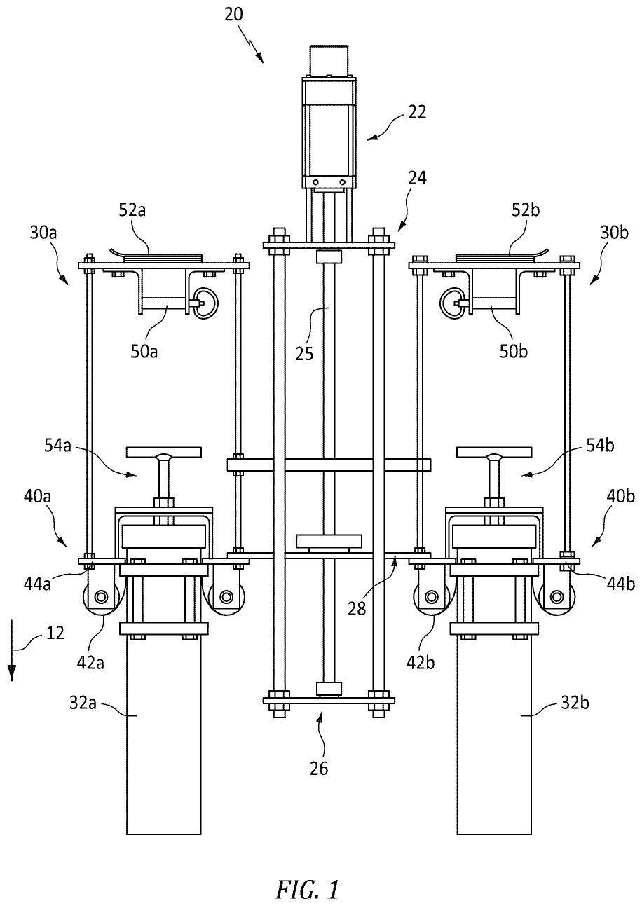

Sediment sampler system 10, FIG. 1, according to one embodiment of the present invention has a stationary frame assembly 20 with a first drive mechanism 22 positioned at the upper portion of a frame 24 and including a lead screw mechanism 25. Stationary frame assembly 20 includes a lower portion with a base 26. A movable elevator plate 28 is driven by first drive mechanism 22 via screw mechanism 25. Travelling frame assemblies 30a, 30b are secured to elevator plate 28 and have core tubes 32a and 32b, respectively, which are independently movable in first, downward direction 12 initially by elevator plate 28 and then by second drive mechanisms 40a, 40b which include constant-force springs 42a, 44a and 42b, 44b, respectively.

Left and right core tubes 32a, 32b are illustrated fully extended after nichrome burn wires 52a, 52b have been triggered, as described in more detail below, to release U-members 50a, 50b to disengage from T-members 54a, 54b to enable springs 42a, 44a and 42b, 44b to drive tubes 32a, 32b to the fully extended and “inserted” position. Suitable constant-force springs having approximately 50 lb force include Part No. 9293K562 available from McMaster-Carr of Robbinsville, New Jersey, USA. The exact force is something that can be refined depending on the hardness of the substrate to be sampled.

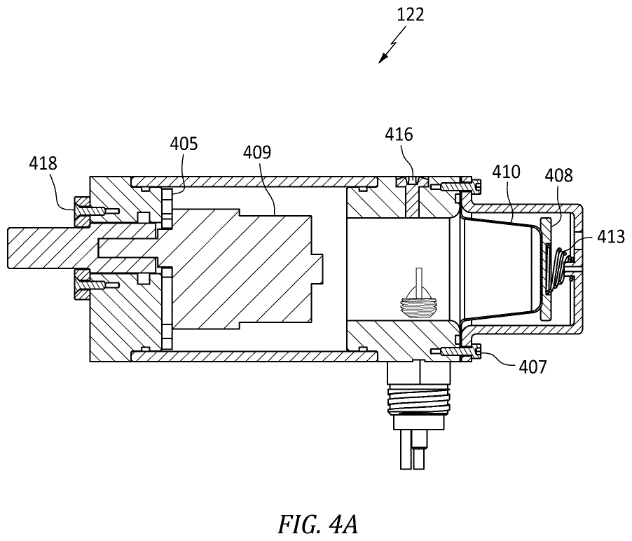

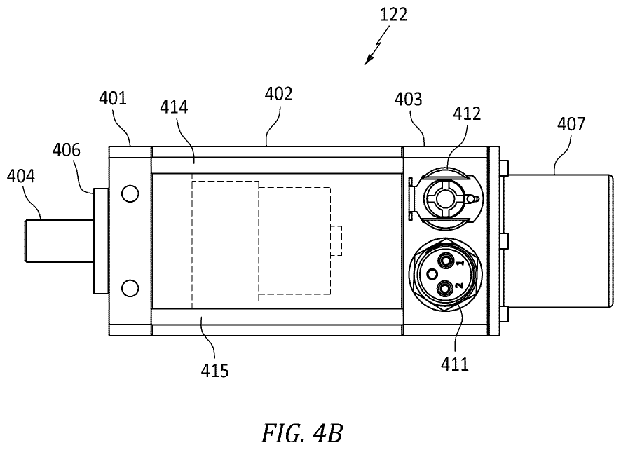

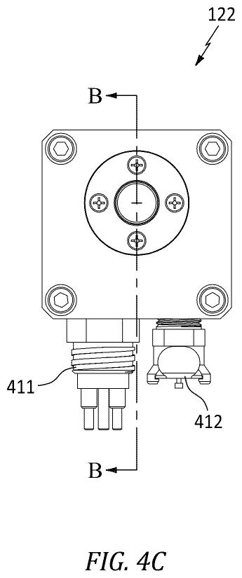

An Autonomous Sediment Core Sampler 100, FIGS. 2-3D, according to another embodiment of the present invention is comprised of four subassemblies: a Stationary Frame Assembly 120, a Traveling Frame Assembly 130a and 130b, an Occlusion Mechanism 160 such as a Core Catcher Assembly having a pivotable plate 162, and a Controller 170 which is a Control Electronics Assembly in some constructions. In one construction according to the present invention, the Stationary Frame Assembly 120 includes two vertically oriented structural components (e.g., U-Channel 6061 Aluminum pieces 121a, 121b) attached to a top and bottom plate to form a rectangular frame. This frame houses and supports the lead screw and drive mechanisms, including a stainless-steel lead screw 125, nylon chain drive sprockets 202 and 204, connecting roller chain (not shown), and an oil-filled actuator drive motor 122 shown in more detail in FIGS. 4A-4C.

The Traveling Frame Assembly 130a, 130b includes a central rectangular frame with a lead screw nut at its center which travels vertically along the lead screw 125 held in the center axis of the stationary frame 120. Bolted on both sides of the central traveling frame there is a core tube holder frame 134a, 136a and 134b, 136b, respectively which provides the structure and alignment guides to support the vertical travel of the respective core sample tube 132a, 132b when powered by two constant-force springs 142a, 144a and 142b, 144b, respectively. The springs reside in coils on rollers immediately adjacent to the core sample tube and connect to the top of the core tube assembly to its passive valve top. Core tube Holder frames are removable from the central Traveling frame and replaceable with different versions to allow for use of various diameter core sample tubes. At the top of the core tube holder frame, there is a lever-activated release mechanism 150a, 150b which holds the springs extended under tension at the highest position of the core sample tube in the armed position. The guide frames around the core sample tube are removable to facilitate removal of a collected sample out of the side of the assembly.

The Occlusion Mechanism 160 is shown in FIGS. 2-3D as a core catcher assembly which is rotatably mounted to the bottom of the stationary frame 120 and provides a plate 161 with elastomeric stoppers such as two rubber stoppers 162a, 162b, formed of natural or synthetic rubber in some constructions, which are used to plug the respective distal (bottom) ends of the core sample tubes 132a, 132b after samples have been collected. The core catcher assembly 160 is attached via a central shaft and is sprung into place, moving from the first position illustrated in FIGS. 2-3D to a second position using a torsion spring with a 90-degree range of motion. At its relaxed state in the second position, the rubber stoppers 162a, 162b align directly below the core sample tubes. The stoppers are attached to the rotating plate via a bolt with wing nut and a slot, allowing the nut to be loosened and the stopper to be removed while still plugging the core sample tube when the tube itself is removed from the assembly with a sample inside.

The controller 170, also referred to as a control module, in one construction has control electronics that are housed in a titanium pressure housing (shown schematically in phantom in FIG. 3C) with a cable (not illustrated) extending at least to drive motor 122 and include a computer, motor controller electronics, and appropriate power conversation electronics. In certain constructions, the controller is embedded in electronics of the vehicle. In some constructions, a tilt detection sensor is included, as described in more detail below. The electronics housing connects via an electrical connector (e.g., 16-pin connector) to an oil-filled junction box which supports connection of multiple autonomous sediment core sampler assemblies as well as a single connection back to the support vehicle platform for power and serial communications. Rubber molded cables provide the connection between the junction box and the actuator motors that are part of each autonomous sediment corer assembly.

In certain constructions, the controller monitors the selected parameter as at least one of (i) a number of revolutions of the first drive mechanism and/or (ii) duration of operation of the first drive mechanism, and then triggers the second drive mechanism when the selected parameter has been achieved. Further, the controller detects when each core tube has been fully extended, such as by monitoring when the second drive mechanism has been triggered, and thereafter commands the first drive mechanism to withdraw the core tube from the sediment in a second direction opposite to the first direction, and then commands the occlusion mechanism to block the opening of that core tube by moving the occlusion mechanism from a first position into a second position. That core tube is then driven in the first direction to block that opening.

Additionally, this type of physical sampling can be paired with AI (artificial intelligence) image processing technology to direct the targeting of the samplers adaptively to locations of highest interest and value. This could also potentially include targeting the sampling of macrofauna utilizing similar sampling methodologies to the coring device proposed here.

In some constructions, one or more tilt detection sensors, located in the present system and/or in the vehicle, are utilized as follows. A first linear actuator such as a lead screw-driven initial insertion mechanism inserts the core tube as far as the softness of the sediment allows. When the tilt sensor detects that the system and/or the vehicle is tilting (being pushed up by the resistance from extending the core rather than the core being inserted deeper into the sediment), tilt detection above a certain magnitude triggers a constant-force spring loaded firing mechanism to drive the core to its full insertion depth. In other constructions, tilt detection informs the system to reposition the tubes to another location because a rock or other dense object has been encountered under the core tube.

In certain constructions utilizing a tilt sensor within the sampler system or within the vehicle itself, the first drive mechanism advances the core tube until a preselected amount of tilt (e.g., 5 degrees, 10 degrees, 15 degrees or 20 degrees) is reached, at which point the controller activates the trigger such as burn wire 52a, 52b, FIG. 1. In other constructions utilizing a tilt sensor, advancement of the core by the first drive mechanism is halted when tilt exceeds the preselected amount, and the controller requests repositioning by the vehicle to sample another location on the sediment. In yet another construction, tilt exceeding a preselected amount during advancement by the second drive mechanism indicates a possible failure in satisfactory core sampling.

One method of using a sampler according to the present invention is as follows. Empty core sample collection tubes are installed into the traveling frame assembly with a passive valve top inserted into the top of the tube and held in place with a hose clamp and the tube guides installed to ensure only vertical motion. At the center of the valve top is an “eye” bolt which provides the location for the release mechanism to attach. The constant-force springs are attached to the sides of the valve top.

Next, each core sample tube is lifted vertically, extending the springs, and the eye bolt is latched into the release mechanism. A safety locking pin is then installed locking the release trigger lever in place and preventing the possibility of “firing” the core under tension accidentally.

The traveling frame is moved into position with the bottom edge of the core sample collection tube located just below the Core catcher plate, allowing the plate to rest, passively sprung, against the side of the tube. At this point, the sampler is considered ready for deployment.

Finally, safety pins are removed immediately prior to deployment to arm the mechanism. The vehicle or lander upon which the sampler is mounted descends to the seafloor to a sampling location.

Seafloor Operation: Once arrived at a seafloor sampling location, the vehicle sends a signal to the control electronics to begin the sampling process. In constructions with two core sample collection tubes per assembly, each sampling event typically collects two sediment core samples using identical mechanisms simultaneously. The sampler controller commands the actuator motor to rotate the lead screw, driving the traveling frame assembly (and core tubes) down to the seafloor with the goal of slowly inserting the tip of the core sample collection tubes 2-10 cm depth, more preferably 3-5 cm depth, into the upper layers of the sediment (core tube length is 3 to 5 time longer than this anticipated insertion depth).

In some constructions, during this initial insertion process, the electronic controller monitors a tilt sensor in real time to detect if the lowering of the core tube is tipping or tilting the vehicle rather than inserting the tubes into the sediment. This could be an indication of a sampling attempt a location unsuitable (e.g., too hard) for coring and present a real time option to abort the sample process prior to triggering and thereby prevent a potentially failed sample.

As the traveling frame assembly descends on the lead screw, the trigger lever arms (FIGS. 2 and 3B) contact the stationary frame and are lifted (rotated) until they move clear of the latching mechanisms, such as releasing eyebolts 152a, 152b, FIG. 3B. This allows the constant-force springs 142a, 144a and 142b, 144b to move the respective core tube 132a, 132b freely downward at greater velocity (e.g., approximately 50 lbs/in2 of spring force).

The springs drive the tube into the sediment to a depth of 20-30 cm in one construction, stopping either when the insertion resistance exceeds that of the substantially constant force being applied by the springs, or when they have reached their full travel. The passive rubber flap valve at the top of the core allows water to flow up and out of the tube itself as this happens.

Once the insertion process is complete, the traveling assembly is signalled to then lift using the lead screw drive to fully remove the core from the seafloor and lift it into a stowed position approximately 40 cm above its full inserted position.

After the bottom edge of the sample collection tubes clears the core catcher plate, the plate passively rotates on its torsion spring into position with the rubber stoppers positioned directly below (and approximately flush with) the core tubes. The lead screw then drives the traveling assembly back down a few centimeters to seat the bottom of the core tubes onto the rubber stoppers 162a, 162b, plugging them closed and preventing sediment from escaping during the remainder of the deployment and recovery.

Eventually the vehicle or lander ascends to the surface and is recovered onto the support vessel. On deck, the core sample collections tubes can be removed for processing by removing the core tube guides, the valve top assembly, and loosening the wing nut that secures the stopper to the core catcher plate. The core is then free to slide sideways away from the assembly, with stopper still in place, to be taken away for processing.

According to one embodiment, each corer assembly consists of a unit of two sediment cores such as illustrated in FIGS. 1 and 2, for example. On a given mission, users can choose to add multiple units to a platform to achieve scaling quantity of samples to ensure adequate replication. For example, the current version of Orpheus AUV could carry four of these units, giving it the ability to collect up to 8 sediment cores autonomously per vehicle deployment, taking samples two cores at a time. The design could be modified in the future to accommodate single core insertions, or perhaps up to three cores at a time, depending on specific mission needs.

FIGS. 4A-4C depict one construction of the actuator assembly 122, with FIG. 4A being a cross-sectional view along lines B-B of FIG. 4C. Illustrated are actuator shaft endcap 401, actuator body tube 402, actuator bladder endcap 403, actuator shaft 404, actuator motor adaptor plate405, actuator shaft thrust ring 406, actuator diaphragm cap 407, actuator diaphragm plunger 408, Servocity 10 RPM Gear motor 409, Bellofram bladder 410, Subconn Micro Circular Series 2 Pin M 411, plastic quick-disconnect 412, conical compression spring 413, hexagon socket head cap screws 414 and 415, sealing pan head screw 416, six hexagon socket head cap screws 417, and four countersunk flat head screws 418. Motor 409 is surrounded with oil within tube 402 to protect it from the liquid environment.

An optimal sediment core sample requires minimizing disruption of the proximal seafloor and therefore minimizes insertion velocity through the upper layers of sediment to mitigate potential disruption of this crucially important area while still being able to achieve 10-20 cm of depth beneath the surface. The undisturbed area of water to sediment interface is of paramount importance to sample analysis for many disciplines. Additionally, the insertion to full depth must be as gentle as possible while still applying necessary forces to minimize compaction of sediment along the inner faces of the sample tube due to friction.

The present design utilizes a novel compound mechanism for positioning and applying the forces necessary to insert a core tube into sediment without compromising the integrity of the sample inside or relying on the vehicle platform carrying the sampler to produce large reaction forces to hold the assembly in place. Existing designs for similar systems have failed to simultaneously address both these driving requirements, making them unsuitable for smaller AUVs and leading to significant limitations in their usage and sample quality. The use of the lead screw for initial slow and steady insertion and subsequent extraction paired with the ability to generate an impulse of force using springs allows this design to utilize the inertia of a vehicle as an opposing “force” rather than a static counter force such as weight or thrust.

The use of constant-force springs specifically versus more standard extension or compression springs is also important as the force required for insertion increases the deeper a core is inserted into the sediment. While also capable of producing the necessary impulse of force, standard springs generate their maximum force at their maximum displacement which would translate into a linear reduction of force as the core tube was pushed lower, which is the opposite of what is desired. Constant-force springs maintain a consistent force throughout enabling the ability for deeper insertion.

The use of springs also offers a level of compliance in sampling that makes it favorable for autonomous use, only inserting as deep as the sediment allows, but also being able to move and adjust independently of the traveling frame when not at full extension.

This autonomous Core Sampler design is for intended for use at any ocean depth, including operational at full-ocean depth at ˜10,900 meters.

The term “portion” as utilized herein refers to a section or region of a component, without necessarily indicating any physical difference between two or more portions apart from location such as “upper portion” and “lower portion”.

Although specific features of the present invention are shown in some drawings and not in others, this is for convenience only, as each feature may be combined with any or all of the other features in accordance with the invention. While there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature.

It is to be understood that the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions. Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on, or executable by, a programmable computer including any combination of any number of the following: a processor, a storage medium readable and/or writable by the processor (including, for example, volatile and non-volatile memory and/or storage elements), an input device, and an output device. The input device and/or the output device form a user interface in some embodiments. Program code may be applied to input entered using the input device to perform the functions described and to generate output using the output device.

Embodiments of the present invention include features which are only possible and/or feasible to implement with the use of one or more computers, computer processors, and/or other elements of a computer system. Such features are either impossible or impractical to implement mentally and/or manually for autonomous vehicles.

Any claims herein which affirmatively require a computer, a processor, a controller, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a controller, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays).

A computer can generally also receive (read) programs and data from, and write (store) programs and data to, a non-transitory computer-readable storage medium such as an internal disk (not shown) or a removable disk or flash memory. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium or other type of user interface. Any data disclosed herein may be implemented, for example, in one or more data structures tangibly stored on a non-transitory computer-readable medium. Embodiments of the invention may store such data in such data structure(s) and read such data from such data structure(s).

It is the intention, therefore, to be limited only as indicated by the scope of the claims appended hereto. Other embodiments will occur to those skilled in the art after reviewing the present disclosure and are within the following claims.

What is claimed is:

1. A system for obtaining a sediment core sample, comprising:
a stationary frame assembly including a first drive mechanism;
at least one traveling frame assembly movable in a first direction relative to the stationary frame and carrying at least one core tube and carrying a second drive mechanism for each core tube;
wherein the first drive mechanism is configured to advance the traveling frame in the first direction until a selected parameter is achieved;
wherein the second drive mechanism is configured to drive the core tube to a fully extended position in the first direction when the selected parameter is achieved, and wherein the second drive mechanism applies a constant force while driving the core tube to the fully extended position;
an occlusion mechanism configured to block the opening of each core tube after the core tube is removed from the sediment; and
a controller configured to activate the first drive mechanism to initiate obtaining the core sample.

2. The system of claim 1 wherein the first drive mechanism includes a linear screw drive.

3. The system of claim 1 wherein the second drive mechanism includes at least one constant-force spring.

4. The system of claim 1 wherein the controller monitors the selected parameter as at least one of (i) a number of revolutions of the first drive mechanism and/or (ii) duration of operation of the first drive mechanism.

5. The system of claim 1 wherein the occlusion mechanism includes a core catcher assembly having a rotatable plate with a stopper for each core tube.

6. The system of claim 1 wherein the controller detects when each core tube has been fully extended and commands the first drive mechanism to withdraw the core tube from the sediment, and then commands the occlusion mechanism to block the opening of that core tube.

7. A sampler system attachable to a vehicle and configured to autonomously obtain a sediment core sample, comprising:
a stationary frame assembly including a first linear drive mechanism;
at least one traveling frame assembly movable in a first direction relative to the stationary frame, each traveling frame assembly carrying at least one core tube and carrying a second drive mechanism for each core tube;
wherein the first drive mechanism is configured to advance at least one of the traveling frames linearly in the first direction until a selected parameter is achieved;
wherein the second drive mechanism is configured to drive the core tube to a fully extended position in the first direction when the selected parameter is achieved, and wherein the second drive mechanism includes at least two constant-force springs to apply a constant force while driving the core tube to the fully extended position;
an occlusion mechanism configured to block the opening of each core tube after the core tube is removed from the sediment; and
a controller configured to activate the first drive mechanism to initiate obtaining the core sample.

8. The system of claim 7 wherein the controller monitors the selected parameter as at least one of (i) a number of revolutions of the first drive mechanism and/or (ii) duration of operation of the first drive mechanism.

9. The system of claim 7 wherein the controller detects when each core tube has been fully extended and commands the first drive mechanism to withdraw the core tube from the sediment in a second direction opposite to the first direction, and then commands the occlusion mechanism to block the opening of that core tube by moving the occlusion mechanism from a first position into a second position, and then driving that core tube in the first direction to block that opening.

10. The system of claim 7 wherein at least one of the sampler system and the vehicle include at least one tilt detection sensor and, when the tilt sensor detects that the vehicle is tilting beyond a selected amount, the controller triggers the constant-force springs.

11. The system of claim 7 wherein at least one of the sampler system and the vehicle include at least one tilt detection sensor and, when the tilt sensor detects that the vehicle is tilting beyond a selected amount at a first sampling location, the controller stops sampling and requests repositioning to another sampling location.

12. A method for obtaining a sediment core sample, comprising:
selecting a sampler system having a stationary frame assembly including a first linear drive mechanism and at least one traveling frame assembly movable in a first direction relative to the stationary frame and carrying at least one core tube and carrying a second drive mechanism for each core tube;
activating the first drive mechanism to advance the traveling frame in the first direction until a selected parameter is achieved;
after the selected parameter is achieved, utilizing the second drive mechanism to drive the core tube to a fully extended position in the first direction by applying a constant force while driving the core tube to the fully extended position; and
blocking the opening of each core tube after the core tube is removed from the sediment.

13. The method of claim 12 further including attaching the sampler system to a vehicle and deploying the vehicle with sampler system in a liquid environment.

14. The method of claim 13 wherein the second drive mechanism includes at least one constant-force spring loaded firing mechanism to drive the core to its full insertion depth using the inertia of the vehicle to react against rather than the vehicle's ability to push back statically through weight or thrust.

15. The method of claim 12 wherein blocking the opening of each core tube includes rotating a push plate to align an elastomeric stopper directly below each filled core tube, and utilizing the linear drive mechanism to drive the bottom of the core tubes into the elastomeric stoppers to plug them closed and prevent sediment from escaping during the remainder of deployment and recovery.
